(19) United States
(12) Patent Application Publication
(10) Pub. No.: US 2023/0317212 A1
(43) Pub. Date: Oct. 5, 2023

(54) Systems and Methods for Generating Ligand Compounds

(71) Applicant: The Board of Trustees of the Leland Stanford Junior University, Stanford, CA (US)

(72) Inventors: Alexander S. Powers, Stanford, CA (US); Helen Yu, Stanford, CA (US); Patricia A. Suriana, Stanford, CA (US); Ron O. Dror, Stanford, CA (US)

(73) Assignee: The Board of Trustees of the Leland Stanford Junior University, Stanford, CA (US)

(21) Appl. No.: 18/184,600

(22) Filed: Mar. 15, 2023

Related U.S. Application Data
(60) Provisional application No. 63/269,392, filed on Mar. 15, 2022.

(51) Int. Cl. G16C 10/00; G16C 20/10; G16C 20/40

(52) U.S. Cl. CPC G16C 10/00; G16C 20/10; G16C 20/40

(57) ABSTRACT

Systems and methods for generating ligand compound structures are provided. A trained computational framework can utilize an initial core ligand compound structure to generate a ligand compound structure by iteratively adding atomic structures. At each iterative step, the computational framework can select a location for adding an atomic structure and can further select which atomic structure is to be added the selected location.

### CROSS-REFERENCE TO RELATED APPLICATIONS

This application claims priority to U.S. Provisional Application Ser. No. 63/269,392, entitled “Systems and Methods for Generating Ligand Compounds” to Alexander S. Powers et al., filed Mar. 15, 2022, the disclosure of which is incorporated herein by reference in its entirety.

### TECHNOLOGICAL FIELD

The disclosure is generally directed to systems and methods to generate ligand compound structures via deep learning computation, including developing drug-like compounds that associate with macromolecules.

### BACKGROUND

A common goal in drug discovery is to identify pharmaceutical compounds that have the potential to treat disease. A typical computational drug discovery platform utilizes a digital library of small molecules and computationally assesses the ability of each molecule to bind within a pocket of a protein macromolecule. Hits are then biochemically assessed for their ability to bind the protein and/or modulate the protein's functions. Top hits identified via biochemical assessment are then optimized chemically by medicinal chemists and then optimized compounds are reassessed via biochemical experimentation.

### SUMMARY

Several embodiments are directed to systems and methods for generating ligand compound structures. In many embodiments, a computational framework is trained using a library of ligand-protein structures that are deconstructed into atomic features in three-dimensional space. In several embodiments, the deconstructed atomic features are utilized in a supervised training to determine the location of adding an atomic structure and which atomic structure to add. In many embodiments, the computational framework is utilized to generate a ligand compound structure by initially providing the framework a starting core structure, and the framework iteratively adds candidate atomic structures at available positions of core structure. In several embodiments, the computational model adds candidate atomic structures by initially selecting a location to attach the atomic structure and then chooses a particular atomic structure to be attached at the selected location. In some embodiments, the computational framework outputs one ligand compound structure having the optimal score at each atomic position assessed. In some embodiments, the computational framework outputs a set of diverse ligand compound structures.

In one implementation, a computational method is for generating a three-dimensional ligand compound structure. The method comprises selecting a target macromolecule structure and a core ligand compound structure. The core ligand compound is putatively capable of associating with the target macromolecule via a chemical interaction. The method further comprises selecting an attachment location on the core ligand structure for addition of an atomic structure. The method further comprises selecting a particular atomic structure to be added at the selected attachment location. The method further comprises generating a three-dimensional ligand compound structure that is the core ligand compound structure with the selected atomic structure added at the selected attachment location.

In one implementation, the selecting of the attachment location is performed utilizing a computational model capable of interpreting three-dimensional data. The model is trained by utilizing known ligand-macromolecule structures that are deconstructed by removing atomic structures.

In one implementation, the computational model is a neural network that is equivariant to rotation and translation.

In one implementation, the attachment location is at a position of a hydrogen atom.

In one implementation, the attachment location is at a position of a double bound or triple bond.

In one implementation, the attachment location is at a position of an atom capable of gaining a charge.

In one implementation, the training is performed with supervision and binary labels are computed for each attachment location.

In one implementation, the binary label computed is whether the hydrogen would be or would not be replaced by the atomic structure in the generated three-dimensional ligand compound structure.

In one implementation, the selecting of the particular atomic structure is performed utilizing a computational model capable of interpreting three-dimensional data. The model is trained by utilizing known ligand-macromolecule structures that are deconstructed by removing atomic structures.

In one implementation, the computational model is a neural network that is equivariant to rotation and translation.

In one implementation, the particular atomic structure that has been selected to be added is an atom selected from: C, O, N, P, S, H, F, Cl, or Br.

In one implementation, the particular atomic structure that has been selected to be added is a small molecular structure selected from: an alkene, an alkyne, a carboxyl group, an amino group, or a ring structure.

In one implementation, the small molecular structure has between 1 and 30 atoms.

In one implementation, the training is performed with supervision and labels can be computed for an additive state in which one label represents the correct additive state and a plurality of labels represent decoy states.

In one implementation, the core ligand compound structure is a known ligand that has one or more atomic structures removed.

In one implementation, the core ligand compound structure is a computationally generated structure that is putatively expected to associate with target macromolecule structure.

In one implementation, the method further comprises iteratively repeating the step of selecting an attachment location, the step of selecting a particular atomic structure, and the step of generating a three-dimensional ligand compound structure, resulting in an addition of an another selected atomic structure to the ligand structure at another selected location at each iteration. The step of selecting an attachment location, the step of selecting a particular atomic structure, and the step of generating a three-dimensional ligand compound structure are iteratively repeated until a final three-dimensional ligand structure is yielded.

In one implementation, the step of selecting an attachment location, the step of selecting a particular atomic structure, and the step of generating a three-dimensional ligand compound structure are iteratively repeated until the generated three-dimensional ligand compound structure reaches a particular atomic weight.

In one implementation, the step of selecting an attachment location, the step of selecting a particular atomic structure, and the step of generating a three-dimensional ligand compound structure are iteratively repeated until a selected number of iterations are performed.

In one implementation, the step of selecting an attachment location, the step of selecting a particular atomic structure, and the step of generating a three-dimensional ligand compound structure are iteratively repeated until the generated three-dimensional ligand compound structure achieves a particular binding affinity for the target macromolecule structure.

In one implementation, the step of selecting an attachment location, the step of selecting a particular atomic structure, and the step of generating a three-dimensional ligand compound structure are iteratively repeated until a computational model predicts no further attachment points on the ligand structure. The computational model is capable of interpreting the 3D space of atomic structures and interactions between ligands and their associated macromolecule.

In one implementation, the method further comprises chemically synthesizing the final three-dimensional ligand structure to yield a chemically synthesized ligand.

In one implementation, the chemically synthesized ligand is utilized in a medicinal formula for treatment of a medical disorder or disease.

In one implementation, the chemically synthesized ligand is utilized as modulator in biochemical experimentation.

In one implementation, the chemically synthesized ligand is utilized in an agricultural product.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a flow diagram of a method to generate ligand compound structures in accordance with various embodiments.

FIG. 2 provides a conceptual illustration of a computational processing system in accordance with various embodiments.

FIG. 3 provides an overview of an example of a computational framework to generate ligand compound structures. Generation begins with a molecular fragment bound to a protein pocket (Step 0). The method sequentially adds fragments to the ligand, connected by single bonds, until the ligand reaches a user-specified goal, such as molecular weight (Step N). Each action is broken down into two steps: location selection and fragment selection. First, to select a location to attach a fragment, potential attachment points are assigned a score by the Growth Vector Model, an equivariant neural network. The model is trained to recognize likely attachment points by learning from curated structures of ligand-protein complexes. After selecting the best scoring location (arrow), a set of candidate structures are generated by sampling fragments and geometries. These candidate structures are scored by the Fragment Scoring Model, which again is an equivariant neural network trained using a dataset of known ligand-protein complexes. The best scoring state is selected (black box) and the process is repeated (Step 1).

FIG. 4 provides a chart that shows the curated dataset of ligands matches the properties of known drugs. Drug-Likeness Score (Quantitative Estimate of Drug-likeness, QED) was calculated for a list of FDA approved small-molecule drugs and for the ligand in the filtered dataset (Curated PDBbind). Results are presented as normalized histograms.

FIGS. 5A and 5B provide structures that show many drug-like ligands are made of up of similar fragments. 75 most frequent fragments in the library obtained from custom fragmentation algorithm applied to drug-like ligands. The depicted fragments are examples of small molecular structure that can be utilized for attachment.

FIG. 6 provides data that shows many drug-like ligands are made of up of similar fragments. Frequency distribution of all fragments in the dataset. A majority of molecules in the dataset can be fully expressed by less than 100 fragment; a library containing the 100 most frequency fragments can be used to fully reconstruct over half the ligands.

FIGS. 7A to 7F data showing the computational framework example learns to score attachment points and fragments in the three-dimensional context of the binding site. FIG. 7A: The framework identifies optimal attachment points for fragments, called growth vectors, by scoring the ligand hydrogen atoms using a learned model. In the images, darker arrows indicate high-scoring attachment points (≥0.5), while lighter arrows indicate low-scoring ones. Comparison with the complete reference ligands shows that the framework often selects the actual attachment points utilized in these ligands. FIG. 7B: To evaluate performance, a precision-recall curve was plotted for the ability of the framework to correctly identify the attachment points used in reference ligands, given intermediate states. The framework achieves performance well above a random baseline (no skill). This data is derived from 700 ligand examples from the test set. FIGS. 7C and 7D: The framework ranks fragments by scoring each structure with a learned model. Examples show the top 3 ranked fragments (with lowest scoring geometries) at left, with a selected lower ranked fragment at right. The native fragment is that found in the reference ligand. The framework can distinguish between fragments that look superficially similar but make different interactions with the pocket. FIG. 7E: The fragment-scoring model was evaluated by measuring how often it ranked the native fragment first (filled bars) or within the top 10 fragments (outlined bars). It was compared to a naïve baseline (Random), a version of the model fine-tuned with weighted examples, and docking scores (Docking). The analysis used 100 ligand-protein complexes from the test set, and error bars indicate the 95% confidence interval obtained from bootstrapping. FIG. 7F: Using the same approach as in FIG. 7E, fragments were ranked and how often the top-ranking fragment recovered the interactions in the native ligand fragment (filled bars) was assessed. It was also assessed whether the top-ranking fragment formed extra interactions not present in the native fragment (outlined bars).

FIGS. 8A and 8B shows that ligands generated by the computational framework example often appear realistic and form key interactions, unlike those generated by other methods. Reference ligands and pockets for two examples from the test set are shown in the left column. A starting fragment was randomly selected from the reference ligand to initiate expansion; the starting fragment is shown in cyan in the middle and right columns. The starting fragments were expanded using the framework to select attachment point and subsequent fragments; the resulting molecules are shown in the middle images. The results were compared to molecules generated using physics-based docking scores (Glide) to iteratively select fragments. Key interactions are highlighted by circles and dotted lines: hydrogen bonds in, pi-pi interactions, and salt-bridges are highlighted.

FIGS. 9A to 9C provide analysis that shows ligands generated with the computational framework example are similar to real drug-like ligands across many physiochemical properties. 100 ligands were generated using the framework, using 100 unique fragment-pocket structures from the test set. The property distributions of the generated ligands were compared to those of ligands generated with a physics-based docking function called Glide (Docking), a naive probabilistic fragment selection function (Random), and reference ligands from the test set. The molecular weight of the reference ligand was used as the molecular weight cutoff to determine when to stop adding fragments for a particular example. Box and whisker plots show the property distribution of the 100 generated ligands, with the box representing the dataset quartiles (median in the center), whiskers showing the extent of the distribution, and outliers represented by diamonds. Docking score represents the Glide docking score obtained after restrained minimization of the ligand. Off-target docking score represents the average docking score of a ligand when docked against the other 99 pockets in the benchmark set. Synthetic accessibility score is a relative measure of the ease of synthesizing the ligand, with lower scores indicating easier synthesis. Unsatisfied h-bond donors correspond to the number of hydrogen bond donors on the ligand that do not form any hydrogen bond with the protein pocket. All other properties are standard molecular descriptors.

FIGS. 10A to 10D provide data showing the computational framework example learns to approximate physics of molecular geometries and key interactions. FIG. 10A: The trained model was used to score a set of ligand-pocket structures that varied only in the dihedral angle of an attached methyl fragment. Dots represent measured scores and the solid line is a smoothed spline curve. The lowest model scores (most favorable) correspond to the staggered conformation, which is the most energetically favorable. The highest scores correspond to the eclipsed conformation which is the least energetically favorable. FIG. 10B: To test the recognition of hydrogen bonds, the distance between a donor (backbone amine) and acceptor (carbonyl) atom was varied and a model score was computed for each structure. The lowest scores corresponded to reasonable distances for hydrogen bonds (approximately 3 Å). As a control, the acceptor (carbonyl) was replaced with a methyl group and the same relationship was not observed. FIG. 10C: To test the recognition of pi-pi interactions, the distance between the centroids of two perpendicular phenyl rings was varied. A minimum score was observed close to a reasonable distance for pi-pi interactions (4 Å). As a control, one ring was replaced with a methyl group. FIG. 10D: To test the recognition of interactions with metal ions, the distance between a carboxylate oxygen and a calcium ion was varied. The lowest scores correspond to the metal coordination distance observed in the experimental structure (2.6 Å). All structures in the figure were curated from the test set.

FIG. 11 shows that the computational framework example fragment scoring model is robust to small perturbations of the input fragment. For three example input fragment, random translations and rotations were applied; each point in the scatter plots corresponds to a particular perturbation for that example. Translation distance is the magnitude of the translation vector applied to the fragment centroid. Rotation angle is the magnitude of the angle the fragment is rotates around the rotation axis (which is also randomly selected). The framework scoring model was then applied for each new input structure to rank the next fragments to add. It was observed whether the top-ranking fragment was unchanged, had a small change in rank, or a large change in rank.

### DETAILED DESCRIPTION

Turning now to the drawings and data, various systems and methods for generating ligand compound structures based on iterative selection of attachment of atomic structures to the ligand compound structures via computational models are described, in accordance with various embodiments. In several embodiments, a computational framework is trained to understand the spatial interaction of ligand compounds and their associated macromolecule. In many embodiments, a computational framework utilizes a trained model in order to determine an attachment location of an atomic structure. In several embodiments, a computational framework utilizes a trained model to determine which atomic structure to attach at the selected attachment point. In many embodiments, the models of the computational framework are trained utilizing a database of structural data of known ligand-protein interactions. In some embodiments, the computational model generates an optimal ligand compound structure. In some embodiments, the computational model generates a variety of ligand compound structures. In some embodiments, a generated ligand compound structure is utilized to synthesize a ligand compound. Synthesized ligand compounds can be assessed for their ability to bind and/or modulate activity of its associated macromolecule. Furthermore, synthesized ligand compound structures can be utilized in a variety applications, such as (for example) within medicinal formulations, as biological research tools, and/or agricultural products.

### Generation of Ligand Compound Structures

Several embodiments are directed to generating ligand compound structures via a computational framework. In many embodiments, the computational framework utilizes an initial core compound and iteratively adds an atomic structure at an available attachment point. In several embodiments, a trained computational model is utilized to determine location of attachment points. In many embodiments, a trained computational model is utilized to select a particular atomic structure at the selected attachment point.

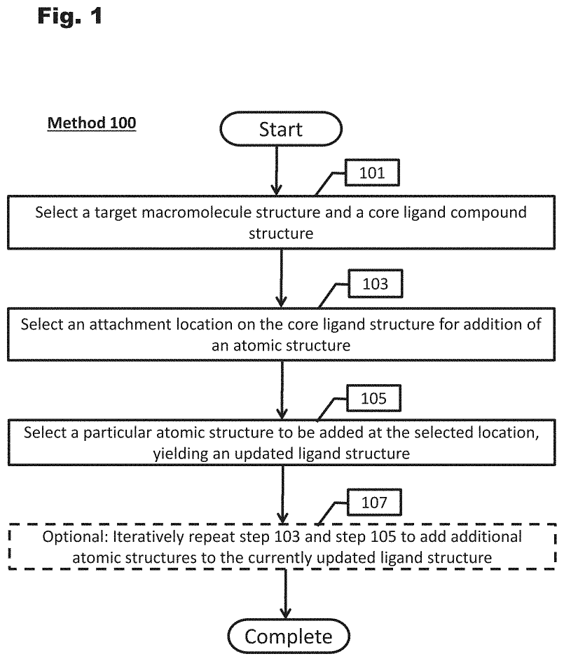

Provided in FIG. 1 is a computational method to generate a ligand compound structure utilizing a computational framework in accordance with various embodiments. Method 100 begins with the computational framework selecting (101) a target macromolecule structure and a core ligand compound structure that associates with the target macromolecule. The target macromolecule is any macromolecule that a user wants to design an associated ligand. In many instances, the macromolecule is a biological macromolecule, such as proteinaceous species, nucleic acids, and other biological polymers. In several embodiments, the macromolecule is a protein (e.g., enzyme) and the associated ligand is designed to modulate the protein's function and/or activity.

In many embodiments, the structure of the target macromolecule is selected, and in particular the structure of the region of ligand association is selected. Often, a ligand associates within a pocket of the protein. In several instances, the protein pocket performs an enzymatic function and the associated ligand is to modulate the function performed by the pocket or the amount of activity performed by the pocket.

A core ligand compound structure is selected. In several embodiments, the core ligand compound is an initial structure of compound putatively capable of associating with the macromolecule structure, which can be any structure putatively having this capability. In many embodiments, the core ligand compound structure is the initial starting structure that will be altered by either iteratively adding one or more atomic structures or modifying one or more of the initial atomic structures of the initial compound structure. In some embodiments, the core ligand compound structure is a ligand already known to associate with its target macromolecule. Examples of ligands with known associations include (but are not limited to) known modulators, drugs, peptides, amino acids, hormones, small proteins and derivations of known modulators. In some instances, a known ligand is stripped down by removing one or more atomic structures, and the stripped-down derivation is utilized as the core ligand compound structure. In some embodiments, the core ligand compound structure is a structure putatively expected to associate with its target molecule, such as (for example) computationally generated structures.

Method 100 selects (100) an attachment location on the core ligand structure for addition of an atomic structure. In many embodiments, a trained computational model selects the location for addition of the atomic structure. Any location of the core ligand structure capable of adding an atomic structure can be selected, such as (for example) locations of hydrogen atoms (e.g., replacing hydrogen atom with an atomic structure), locations of multi-bonding (e.g., replacing a double bond or triple with one or more atomic structures and a single bond or double bond), and locations capable of gaining a charge (e.g., replacing a neutral nitrogen atom with an atomic structure and a positively charged nitrogen atom).

To select a location for atomic structure addition, in accordance with several embodiments, a trained computational model is used. Any computational model capable of interpreting three-dimensional data, and more specifically capable of interpreting the 3D space of atomic structures and interactions between ligands and their associated macromolecule can be utilized. Examples of computational models that can be utilized include (but are not limited to) neural networks, decision trees, random forests, support vector machines (SVMs), nearest neighbors, and naïve Bayes. In some embodiments, the computational model is a neural network that is equivariant to rotation and translation. In some embodiments, the computational model is a tensor field network (e.g., an E(3) equivariant neural network), a SE(3)-equivariant network (e.g., Cormorant), a 3D convolutional neural network (3DCNN), a graph neural network (GNN), or a PointNet, which are capable of interpreting 3D point clouds such that relative position of atoms in the macromolecule-ligand complex can be represented precisely. For more details on an example of an architecture for a model to select the location for addition of the atomic structure, see the Examples section.

In several embodiments, the model for selecting the location for addition of the atomic structure is trained utilizing a collection of macromolecule-ligand structures. For example, the model in the example in described herein utilized the PDBbind dataset. In some embodiments, the dataset for training is filtered to remove common biomolecule ligands or ligands of greater than a selected molecular weight, such that the dataset is enriched for small-molecule ligands. In several embodiments, the macromolecule-ligand structures are deconstructed into atomic structures. The computational model can learn how to generate a ligand by reverse-engineering the ligand from its full natural state into its minimally sized state by sequentially removing its atomic structures.

Several embodiments are directed to training a model for selecting the location for addition of the atomic structure. To do so, in accordance with various embodiments, the computational model can be trained to generate a ligand in its full natural state from its minimally sized state. In some embodiments, the training is performed with supervision, and binary labels can be computed for each location capable of adding an atomic structure. For instance, a binary label can be computed for each ligand hydrogen atom in each intermediate state, corresponding to whether the hydrogen would be or would not be replaced by an atomic structure in the generated three-dimensional ligand compound structure, resulting in a trained model capable of selecting a location for adding an atomic structure by replacing the hydrogen atom at that selected location.

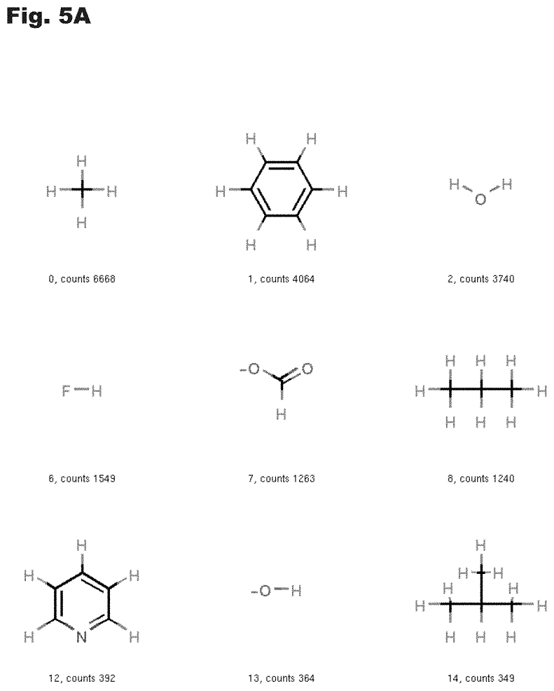

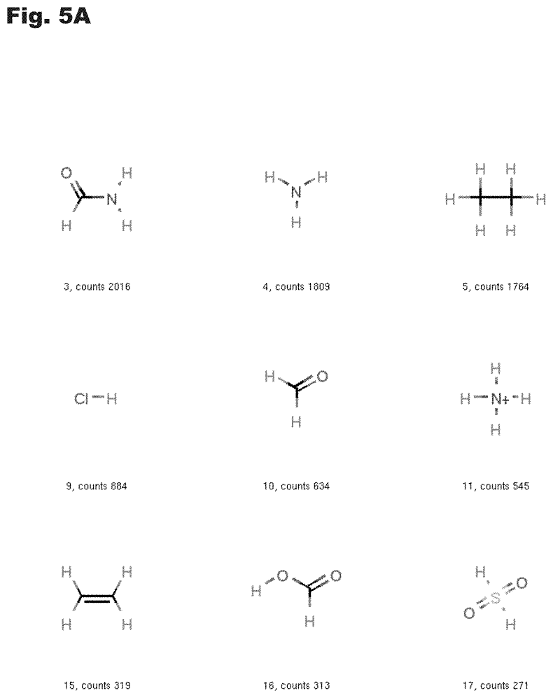

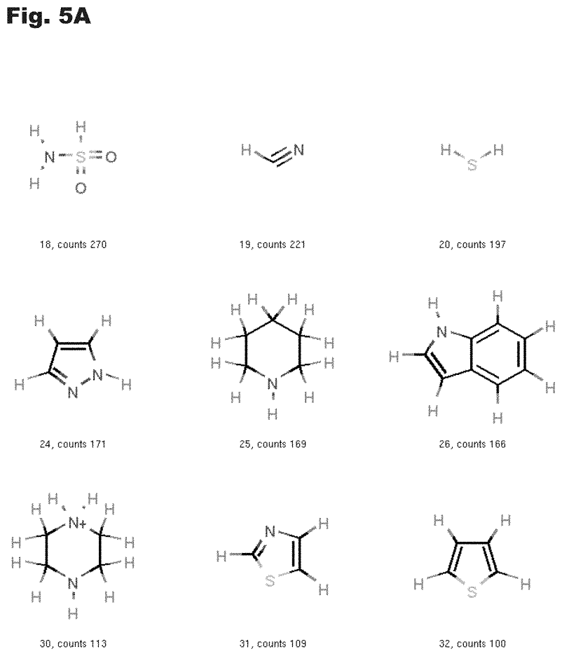

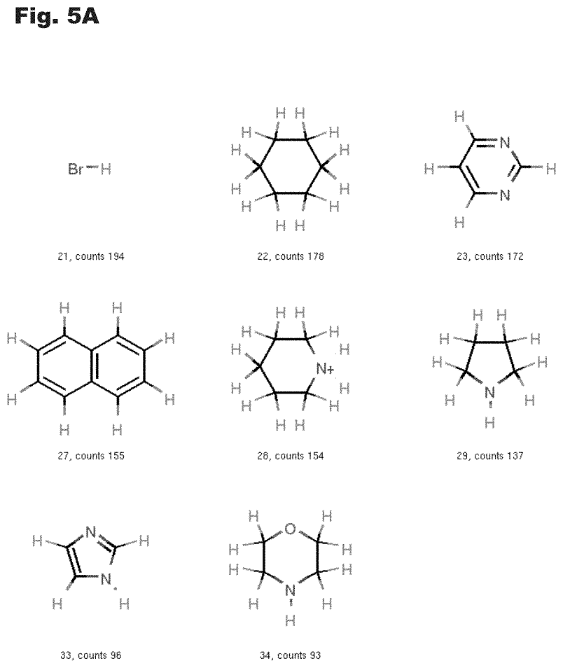

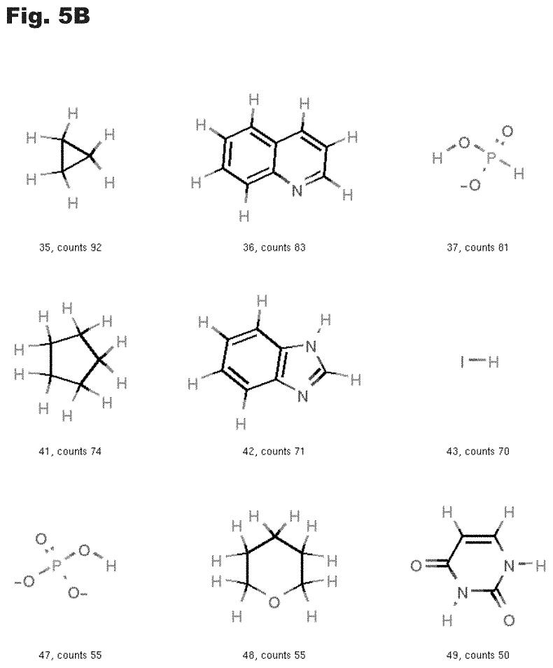

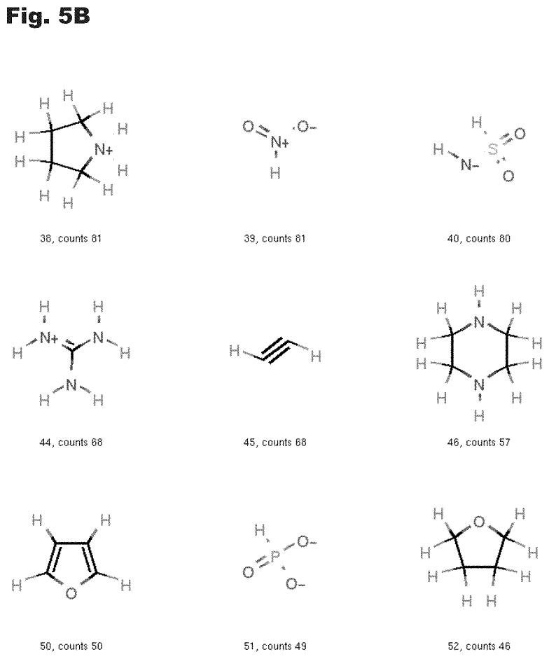

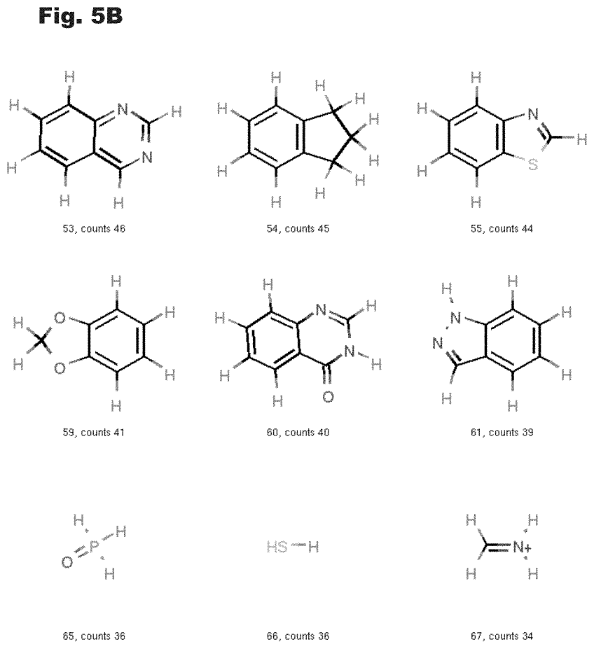

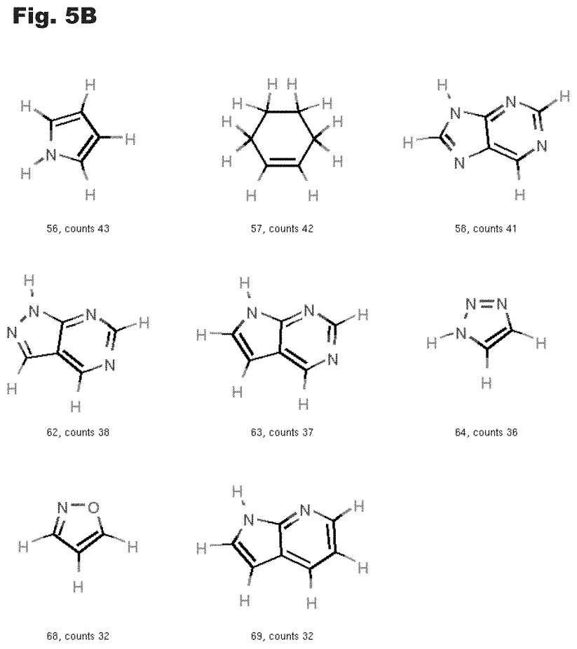

Method 100 also selects (105) a particular atomic structure to be added at the selected location, generating a ligand structure with the selected atomic structure added at the selected location. In many embodiments, a trained computational model selects the particular atomic structure to be added. Any atomic structure useful in a small ligand compound can be added atomic structure can be added, such as (for example) atoms and small molecular structures. Typical atoms that can be added include (but are not limited to) C, O, N, P, S, H, F, Cl, and Br. Examples of small molecular structures that can be added include (but are not limited to) alkene, alkyne, carboxyl groups, amino groups, and ring structures. Further examples of small molecular structures that can be added are shown in FIGS. 5A and 5B. In some embodiments, the small molecular structure has between 1 and 30 atoms.

To select a particular atomic structure to be added, in accordance with several embodiments, a trained computational model is used. Any computational model capable of interpreting three-dimensional data, and more specifically capable of interpreting the 3D space of atomic structures and interactions between ligands and their associated macromolecule can be utilized. Examples of computational models that can be utilized include (but are not limited to) neural networks, decision trees, random forests, support vector machines (SVMs), nearest neighbors, and naïve Bayes. In some embodiments, the computational model is a tensor field network (e.g., an E(3) equivariant neural network), a SE(3)-equivariant network (e.g., Cormorant), a 3D convolutional neural network (3DCNN), a graph neural network (GNN), or a PointNet, which are capable of interpreting 3D point clouds such that relative position of atoms in the macromolecule-ligand complex can be represented precisely. For more details on an example of an architecture for a model to select the location for addition of the atomic structure, see the Examples section.

In several embodiments, the model for selecting the atomic structure is trained utilizing a collection of macromolecule-ligand structures. For example, in the model of the example described herein utilized the PDBbind dataset. In some embodiments, the dataset for training is filtered to remove common biomolecule ligands or ligand greater than a selected molecular weight, such that the dataset is enriched for small-molecule ligands. In several embodiments, the macromolecule-ligand structures are deconstructed into atomic structures. The computational model can learn how to generate a ligand by reverse-engineering the ligand from its full natural state into its minimally sized state by sequentially removing its atomic structures.

Several embodiments are directed to training the model for selecting the atomic structure to be added. To do so, in accordance with various embodiments, the computational model can be trained to generate a ligand in its full natural state from its minimally sized state. In some embodiments, the training is performed with supervision, and labels can be computed for an additive state in which one label represents the correctly additive state and a plurality of labels represents decoy states. The decoy states can be randomly sampled incorrect additions of atomic structures. For instance, the correctly additive state can be assigned a label of 1 and the decoy example states can be assigned in a label of 0, resulting in a trained model capable of selecting a particular atomic structure to be added.

Method 100 also optionally iteratively repeats (107) step 103 and step 105, resulting in an addition of another selected atomic structure to the ligand structure at another selected location at each iteration. In some embodiments, step 103 and step 105 are iteratively repeated until a final ligand structure is yielded. In some embodiments, step 103 and step 105 are iteratively repeated until the generated ligand compound structure reaches one or more metrics. For instance, in some embodiments, step 103 and step 105 are iteratively repeated until the generated ligand compound structure reaches a particular atomic weight, until a selected number of iterations are performed, and/or until the generated ligand compound structure achieves a particular binding affinity for the macromolecule. In some embodiments, step 103 and step 105 are iteratively repeated until a computational model predicts no further attachment points on the ligand structure; a computational model capable of interpreting the 3D space of atomic structures and interactions between ligands and their associated macromolecule can be utilized, such as the model described in reference to step 05.

While specific examples of methods for generating a ligand compound structure are described above, one of ordinary skill in the art can appreciate that various steps of the process can be performed in different orders and that certain steps may be optional according to some embodiments of the disclosure. As such, it should be clear that the various steps of the method could be used as appropriate to the requirements of specific applications. Furthermore, any of a variety of methods generating a ligand compound structure appropriate to the requirements of a given application can be utilized in accordance with various embodiments of the disclosure.

Once a ligand compound structure is generated, in accordance with various embodiments, the compound is chemically synthesized. The compound to be synthesized can be a final ligand structure or any intermediate ligand structure. Generally, standard chemistry synthesis methods can be utilized. In some situations, synthesis protocols can be generated to produce the generated ligand compound structure. For more on compound synthesis, see, e.g., S. L. Schreiber, Proc Natl Acad Sci USA. 2011 Apr. 26; 108(17):6699-702; J. Li, et al., Science. 2015 Mar. 13; 347(6227):1221-6; and J. W. Lehmann, et al., Nat Rev Chem. 2018 Feb.; 2(2):0115; the disclosures of which are each incorporated by reference.

Once the compound is synthesized, it can be utilized in a variety of applications. For instance, a synthesized ligand compound can be utilized in a medicinal formula for treatment of a medical disorder or disease. In some situations, a synthesized ligand compound can be utilized as modulator in biochemical experimentation. In some situations, a synthesized ligand compound can be utilized in an agricultural product, such as (for example) an herbicide or a pesticide.

### Computational Processing System

A computational processing system to generate ligand compound structures in accordance with various embodiments of the disclosure typically utilizes a processing system including one or more of a CPU, GPU and/or other processing engine. In some embodiments, the computational processing system is housed within a computing device or a set of connected computing devices such as (but not limited to) a computer, mobile phone, a tablet computer, and/or portable computer. A set of computing devices can be connected in any manner that allows data communication, such as (for example) a wired connection, Bluetooth, Wi-Fi, a cellular system, a cloud system, or an internet modem connection. In certain embodiments, the computational processing system is implemented as a software application to be performed on a computing device.

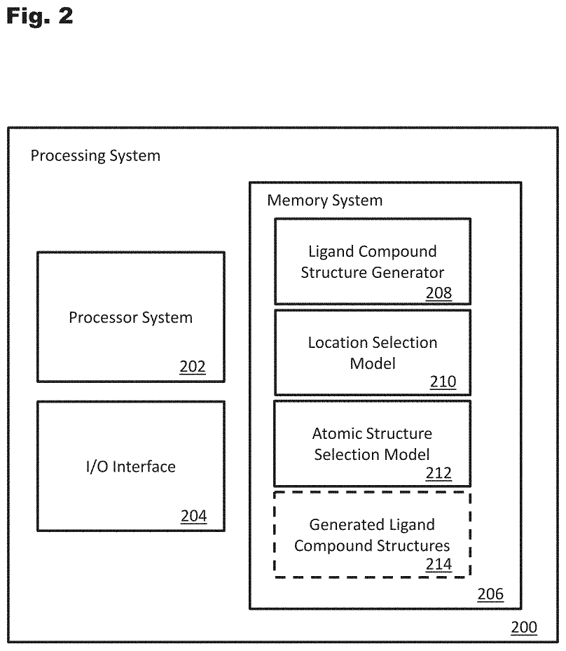

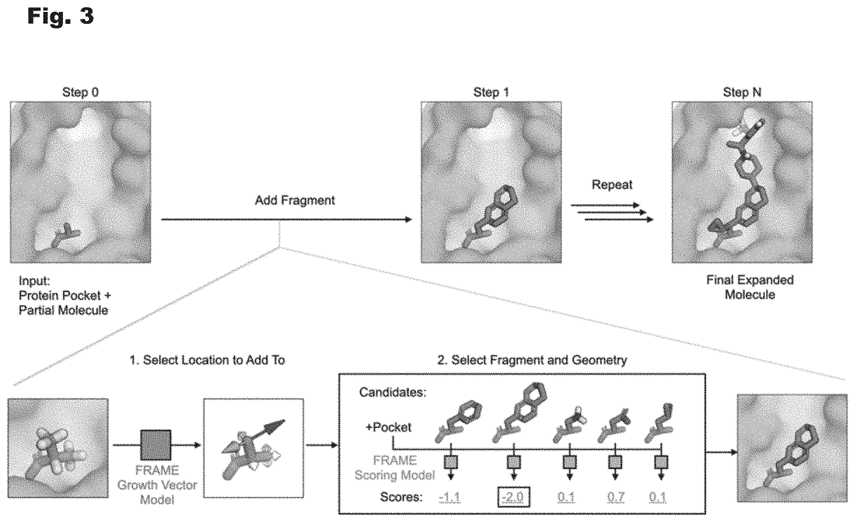

A computational processing system in accordance with various embodiments of the disclosure is illustrated in FIG. 2. The computational processing system 200 includes a processor system 202, an I/O interface 204, and a memory system 206. As can readily be appreciated, the processor system 202, I/O interface 204, and memory system 206 can be implemented using any of a variety of components appropriate to the requirements of specific applications including (but not limited to) CPUs, GPUs, ISPs, DSPs, wireless modems (e.g., Wi-Fi, Bluetooth modems), serial interfaces, depth sensors, IMUs, pressure sensors, ultrasonic sensors, volatile memory (e.g., DRAM) and/or non-volatile memory (e.g., SRAM, and/or NAND Flash). In the illustrated embodiment, the memory system is capable of storing a ligand compound structure generator application 208, which can include a location selection model 210 and an atomic structure selection model 212. The ligand compound structure generator application 208 can be downloaded and/or stored in non-volatile memory. When executed the ligand compound structure generator application 208 is capable of configuring the processing system to implement computational processes including (but not limited to) the computational processes described above and/or combinations and/or modified versions of the computational processes described above. In several embodiments, the ligand compound structure generator application 208 generates ligand compound structures 214, which can optionally be stored in the memory system. In certain embodiments, the ligand compound structure generator application 208 utilizes model parameters, which can be stored in memory to perform processes including (but not limited to) selecting a location to add an atomic structure and selecting the particular atomic structure to add.

While specific computational processing systems are described above with reference to FIG. 2, it should be readily appreciated that computational processes and/or other processes utilized in the provision of ligand compound structure generation in accordance with various embodiments of the disclosure can be implemented on any of a variety of processing devices including combinations of processing devices. Accordingly, computational devices in accordance with embodiments of the disclosure should be understood as not limited to specific computational processing systems and/or ligand compound structure generator systems. Computational devices can be implemented using any of the combinations of systems described herein and/or modified versions of the systems described herein to perform the processes, combinations of processes, and/or modified versions of the processes described herein.

### Examples

The embodiments of the disclosure will be better understood with the various examples provided within. Provided within is a description of a system and method as an example of performing the various embodiments as described. In this particular example, a fragment-based molecular expansion framework, FRAME, was developed to generate ligands in an iterative fashion. The FRAME process performs a sequence of steps in 3D space, where new molecular fragments are attached to the growing seed molecule. E(3) equivariant neural networks were employed to predict and score actions from states represented as 3D atomic point clouds, which allows incorporation of the precise geometry of both the intermediate ligand and protein pocket without separate encoding steps. The framework generates only valid molecules that follow chemical rules such as proper valence. Despite the limited training data (less than 4000 protein-ligand pairs), the framework creates realistic ligands with key interactions. Furthermore, the approach is interpretable as the framework's individual actions align with chemical intuition and physical principles. Interpretability is critical in generating promising candidate molecules that are of practical use to medicinal chemists. These results demonstrate the potential of the learning framework and associated datasets for diverse tasks in ligand optimization.

Expanding Molecular Structures with Learned Models

The core of FRAME is an agent which selects actions to expand a ligand molecule based on the current molecular state. Initially, the state comprises the 3D atomic structure of the seed fragment as well as the protein pocket, including atom locations, elements, and bonds (FIG. 3). The agent sequentially adds fragments to the ligand, connected by single bonds, until a user-specified goal is reached. The fragments are selected from a user-specified library whose composition can be varied without model retraining; for benchmarking a library of the 60 most common fragments was utilized. Each action is broken down into two steps: first, selecting a location to attach a fragment and second, choosing which fragment to add and the attachment geometry. Two separate models that utilize E(3) equivariant neural networks were trained to make predictions for each step (FIG. 3).

The FRAME models are trained using a curated set of protein-ligand structures from the PDBbind database; each ligand structure is then broken down into a sequence of learnable actions, called trajectories. As each ligand in the dataset is synthesizable and binds to its corresponding target, its structure and constituent fragments are to some extent optimized and desirable compared to a random molecule. The models were trained to reconstruct the trajectories of these known active ligands, as a proxy for training on “optimal” ligands. Although this approach may or may not necessarily output the single best ligand, an initial goal is to produce realistic candidates for consideration by medicinal chemists or other algorithms. Thus, the challenge is for the models to learn the generalizable rules (favorable interactions, geometry, synthetic feasibility) that produced these ligands, rather than simply memorizing them.

To train models to select locations, fragments, and geometries, the molecule expansion trajectories were treated as a supervised learning problem using state-action pairs. For the location selection step, ligand hydrogen atoms were used as attachment points for new fragments. The FRAME growth vector model is trained as a binary classifier to determine whether each ligand hydrogen atom should be used as an attachment point or not. Using the trajectories, binary labels were computed for each ligand hydrogen atom in the intermediate states, corresponding to whether this hydrogen would be replaced by a fragment in the final state.

For the fragment selection step, the FRAME fragment scoring model outputs a single score given a ligand with candidate attached fragment and the protein pocket. Candidates structures are generated by enumerating fragments from the library, attachment points on the fragments, and dihedral angles to a specified resolution. The model scores are used to rank the candidate states. The model is trained using intermediate states (the “native” fragment) and decoy states with randomly sampled other fragments or geometries, labeled as 1 and 0 respectively. Several fine-tuning improvements to this model were explored; such as more sophisticated decoys selected by the partially trained model and weighted the examples depending on the types of interactions formed with the protein pocket.

This process can be run to output a single ligand, by greedily choosing the highest scoring actions at each step. Alternatively, the model scores can be used as heuristics to inform more sophisticated or stochastic search strategies that output a set of diverse ligands. This example focuses on the greedy case. The molecule expansion can continue until a user-specified goal is reached, such as molecular weight or number of atoms. Alternatively, FRAME can automatically detect an end point when the location attachment model outputs no predicted attachment points.

### Datasets

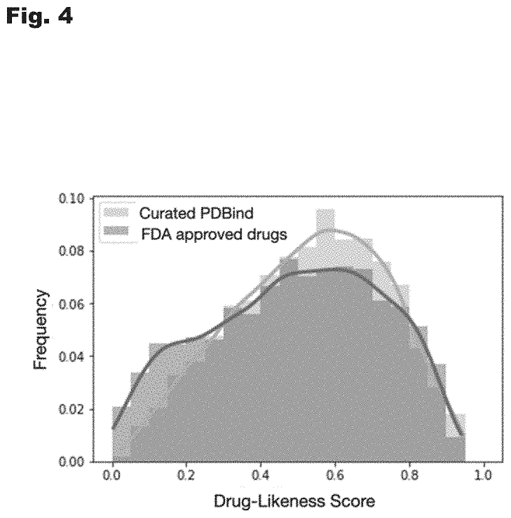

Ligand-protein complexes were derived from the PDBbind refined dataset, a collection of high-resolution 3D structures. The dataset was filtered to remove common biomolecules (lipids, peptides, carbohydrates, and nucleotides), duplicate ligands, and compounds outside a molecular weight range. This resulted in a dataset of 4200 ligands, with drug-likeness scores similar to those of FDA approved drugs (FIG. 4). Trajectories were created by sequentially removing fragments from each ligand until 25% of the heavy atoms were remaining. Two datasets were then derived: a dataset used to evaluate the location to add fragments and a dataset to evaluate candidate fragments. Both datasets were split using the same split of protein-ligand pairs into training (70%), validation (15%), and test (15%) sets. To prevent data leakage, these structures were split such that no proteins had more than 30% sequence identity with proteins in the other sets. The pocket structures were also prepared to prevent leakage of information about the reference ligand but mimic a realistic design scenario. Each reference ligand atom was shifted 2.5 Å in a random direction and the pocket was defined as all residues within 6 Å of any noised ligand atoms.

The fragment library was derived by applying a graph fragmentation process to the ligand dataset combined with a manually curated set of small functional groups. The fragments mainly consist of minimal functional groups (carboxy, methyl, fluoro etc.) and diverse rings (FIGS. 5A and 5B). Differing protonation and tautomeric states are tracked. The dataset contains 900 unique fragments, though the vast majority of these occurred very rarely (FIG. 6). The model training used all available fragment types in the training set. For constructing full ligands, a subset of the 60 most frequent fragments was sampled as a tradeoff between efficiency and expressivity; these 60 fragments can fully construct a majority of the ligands in the dataset.

### Architecture and Training

To predict actions from atomic structures, E(3) equivariant neural networks were used, which act on 3D points clouds. Each atom in 3D is associated with a feature vector consisting of the element type (C, O, N, P, S, H and F/Cl/Br) and flags indicating whether an atom belongs to the ligand, protein, or the candidate fragment when applicable. The atom-wise feature vectors are updated through the embedding unit layers by aggregating local information of the nearest 50 neighboring atoms. The embedding process also utilizes the relative vectors between atoms through spherical harmonic filters, thus encoding their relative geometry. For scoring fragments, features of fragment atoms are aggregated and passed through fully connected (FC) layers. For the attachment location model, feature vectors of ligand hydrogen atoms are passed through FC layers, which produces a vector of scores corresponding to each hydrogen atom.

### FRAME Learns to Score Attachment Points and Fragments in the Three-Dimensional Context of the Binding Site

Before applying the trained models to generate full ligands, model performance on individual action selection tasks was assessed.

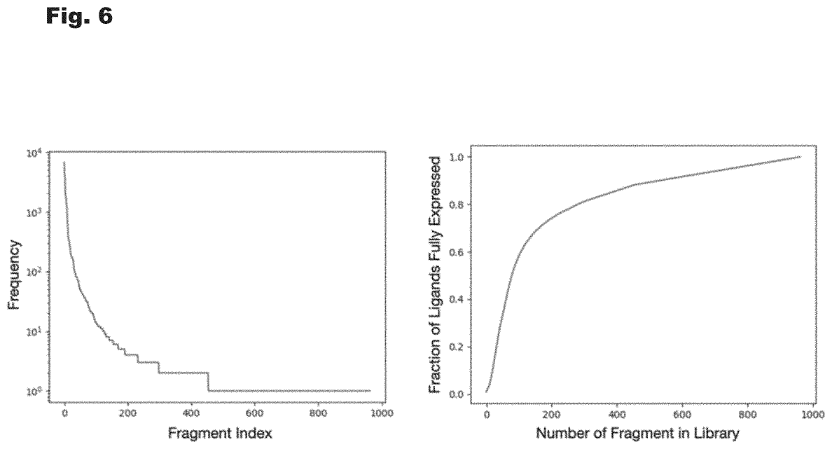

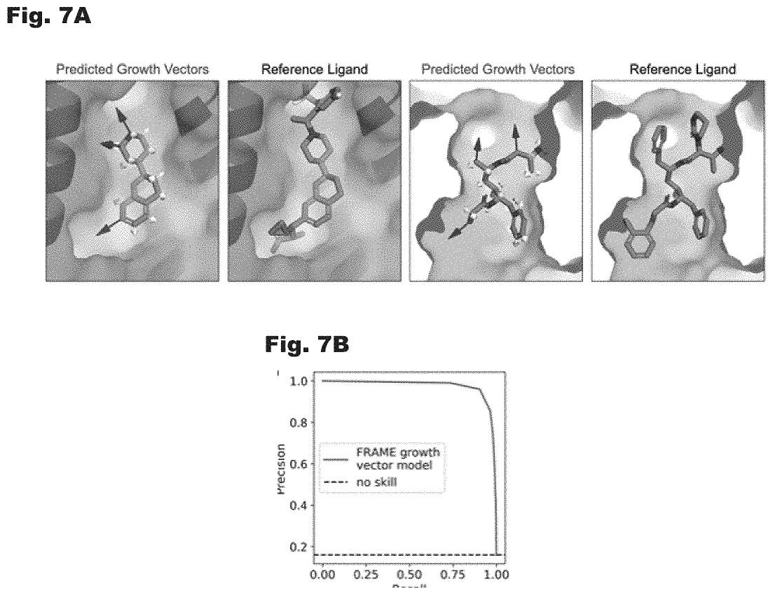

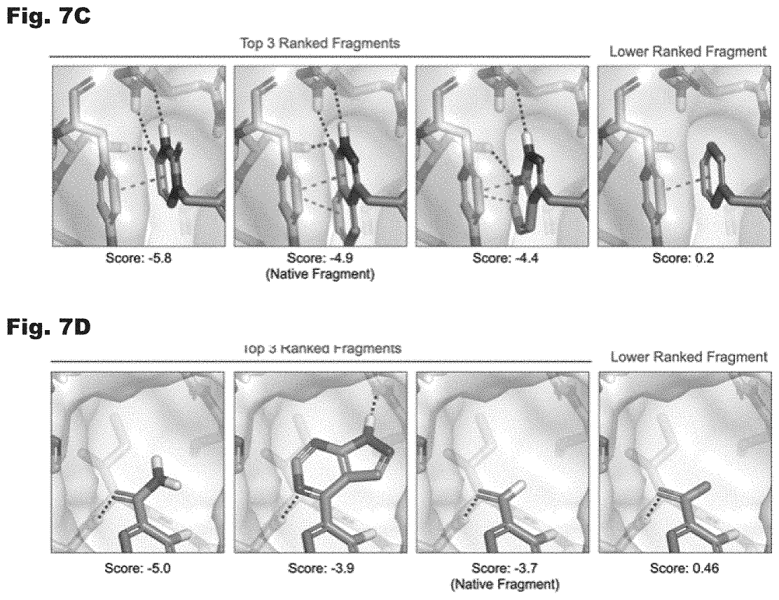

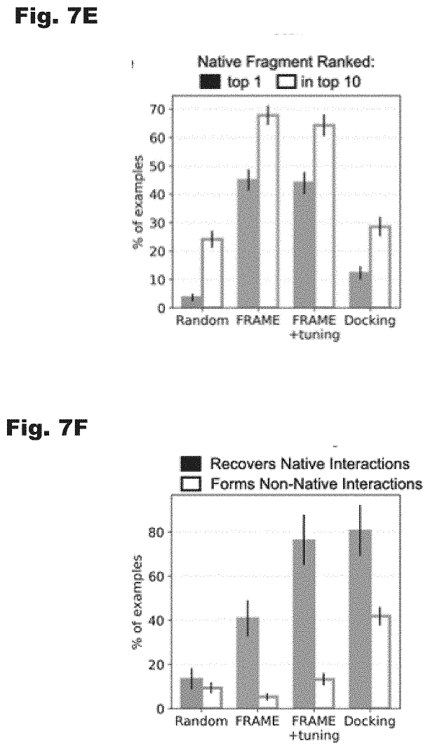

First, it was found that the FRAME growth vector model selected appropriate locations to attach fragments given an intermediate state in the expansion trajectory. From a visual inspection, the model frequently identified unobstructed attachment locations that pointed towards unfilled areas within the protein pocket (FIG. 7A). FRAME often selects the actual attachment points utilized in the reference ligands. Quantitatively, 95% of the points selected by the model are actual attachment points in reference ligands (the model precision) and 92% of the total reference attachment points are selected by the model (the model recall). The ligands used for this evaluation were from the test set; the model was not trained on these ligands or pockets. The FRAME model far outperforms a random baseline (FIG. 7B) and overall, this evaluation suggests that the model has learned transferable skills for this task.

FRAME considers the atoms of both the partial ligand and protein pocket when predicting attachment locations. Information from the ligand atoms may allow FRAME to learn some rules of chemical synthesizability, while the pocket informs steric effects and interactions. To test the relative importance of the pocket information, the growth vector model was trained only on the partial ligand atoms. The performance was somewhat degraded (recall 70%, precision 80%) though still better than the random baseline, indicating the importance of both types of conditioning information.

Next, the FRAME fragment scoring model on fragment selection tasks was evaluated, where the model was tasked with ranking a list of fragments and geometries. Visually, it was found that the model often ranked reasonable fragments and geometries at the top (FIGS. 7C and 7D). For example, in FIG. 7C, the model selected ring fragments that formed both pi-pi interactions and multiple hydrogen bonds—important interactions found in the reference ligand. Notably, the model correctly distinguished between these heterocycles and a phenyl ring that looked superficially similar but could not form the same hydrogen bonds. The model was also capable of enriching diverse fragments. In FIG. 7D, for example, the top three fragments differ in size and chemistry, but all form the same key hydrogen bond with the pocket. This capability for multimodal sampling is important to eventually produce diverse candidates. It was also observed that the model may consider the fragment's potential for further growth vectors. For example, a carboxylate fragment that blocked extension into the larger hydrophobic pocket was ranked significantly lower than an aldehyde, even though they formed the same hydrogen bond.

The ability to enrich fragments found in the reference ligands (termed native fragments) over random fragments is an important quantitative measure of the model's learned ability. On test set examples, the fragment-scoring model selects the native fragment as the top choice 45% of the time, and within the top 10 fragments 65% of the time, which is about three times higher than random choice or using docking scores (FIG. 7E). However, not all fragments are equally important for a ligand's binding affinity; key interactions such as salt bridges are often essential for a functional effect. Thus, the model's ability to select fragments that recover the interactions of the native fragment was measured; the first version of the model recovered 40% of the interactions (FIG. 7F). This was improved by fine-tuning the model with examples weighted by types of interactions, resulting in a 75% interaction recovery rate while avoiding excessive additional interactions (FIG. 7F). In contrast, docking scores tend to overemphasize fragment interactions and often select fragments that produce interactions not formed by the native fragment (40% of examples vs. 10% for FRAME).

### Ligands Generated by FRAME Often Appear Realistic and Form Key Interactions

Generating larger ligand structures requires iterative application of the attachment and fragment selection models, a substantially harder task than scoring single fragments. To evaluate FRAME performance for this task, iterative action selection was performed to generate 100 ligands for 100 unique test protein pockets, using randomly selected small fragments from the reference ligands as starting points. Addition of fragments was stopped when the number of heavy atoms in the ligand passed 90% of the heavy atoms in the corresponding reference ligand. For comparison, ligands were also generated using a state-of-the-art physics-based scoring function (Glide) to iteratively select actions in place of the learned models.

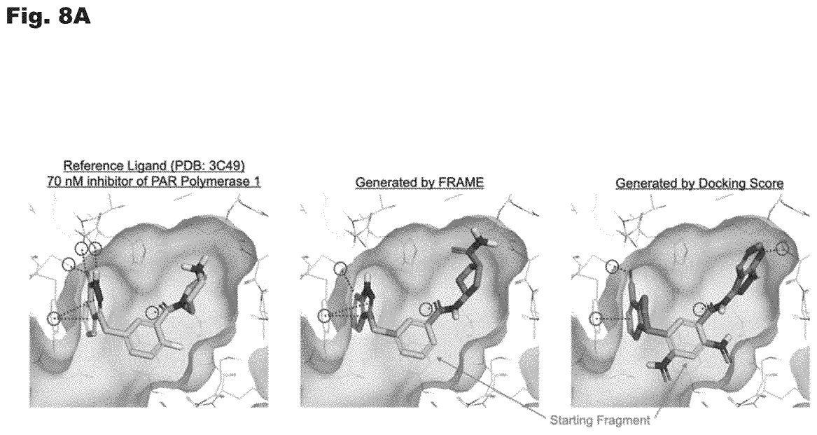

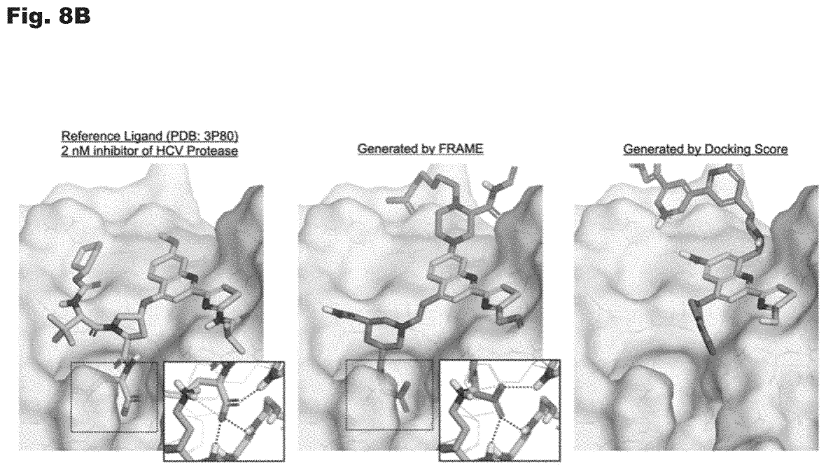

FRAME often generates chemically reasonable ligands that fit the pocket and satisfy key interactions, as demonstrated in several case studies (FIGS. 8A and 8B). For example, small molecule inhibitors of Poly(ADP-ribose) Polymerase (PARP) are of interest for cancer therapy. Starting with a single ring and the PARP structure, FRAME was able to construct a ligand that makes several interactions known to be important for affinity (FIG. 8A). Specifically, a heterocyclic ring extends into a deep cleft to make pi-pi interactions and hydrogen bonds, while an amide links to an aliphatic heterocyclic ring that occupies a shallow pocket. These features are also present in the reference ligand. FRAME also adds an amide group that extends further upward into the pocket, into an area occupied by a water molecule in the crystal structure. In contrast, docking generates a molecule with several issues (FIG. 8A). The ring on the left is added too early, failing to extend fully into the deep cleft, while an aromatic ring is added on the right, failing to fully occupy the shallower pocket. Additionally, several extraneous fragments are added that increase synthetic complexity without adding apparent benefit.

In a more challenging case study, HCV protease presents a mostly shallow, solvent-exposed binding site (FIG. 8B). The initial input fragment is distant from the catalytic site needed for high affinity and inhibition, requiring several precisely placed fragments to reach it. Surprisingly, FRAME was able to expand toward the active site and place a carboxylate fragment in an optimal location to form several hydrogen bonds with the catalytic site residues. In contrast, the ligand extended using docking functions fails to enter the catalytic site at all. Growing a ligand with trained models apparently benefits from the directional awareness absent in docking functions. Interestingly, FRAME also extends the ligand in a direction not occupied by the reference ligand, where it makes an additional salt bridge interaction.

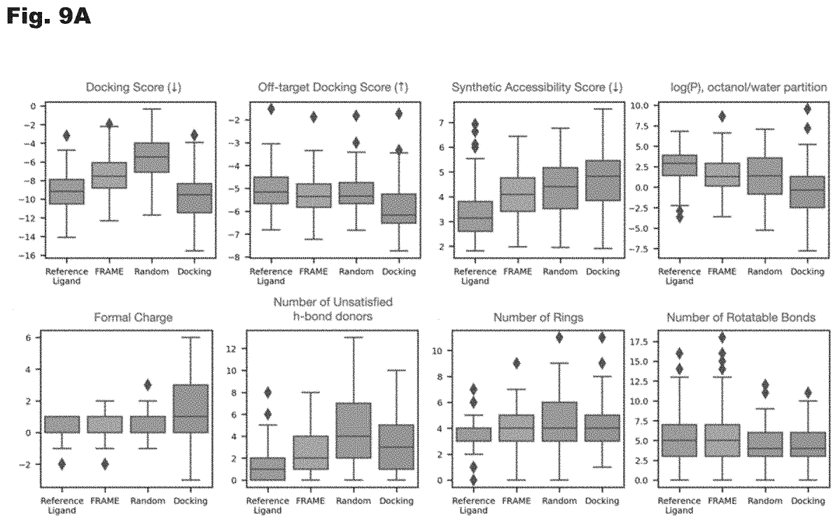

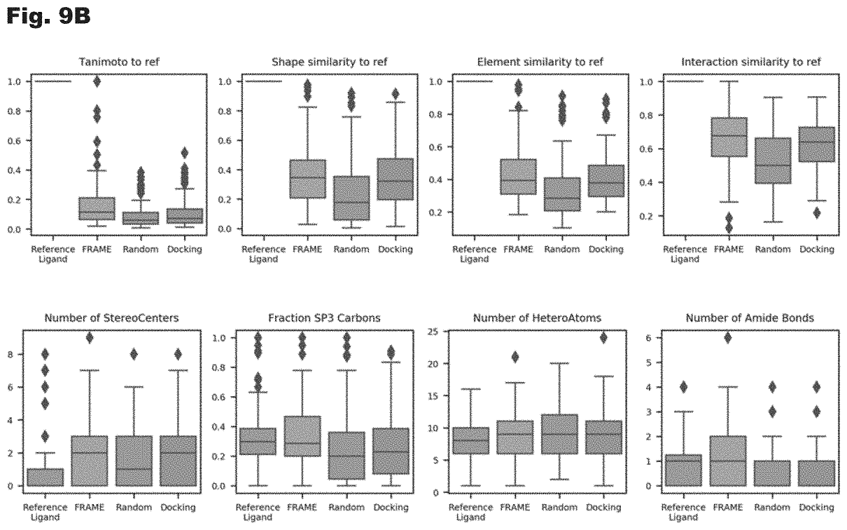

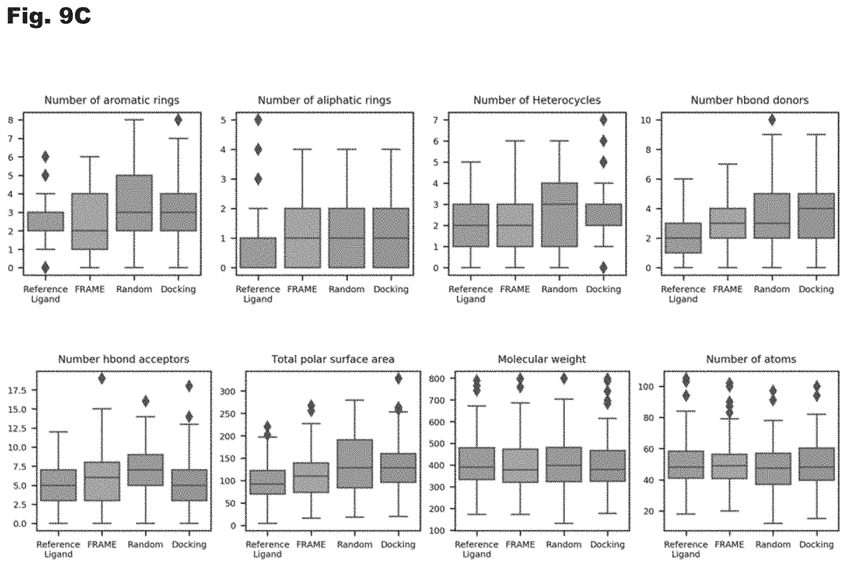

The properties of ligands generated by FRAME were found to match reference drug-like ligands across many key features. As compared with a reference ligand, ligands generated with docking, and against a naïve probabilistic approach (“random”) in which fragments were randomly added if they did not clash with the pocket or form unstable bonds. A panel of 24 properties were measured including ligand-only features such as log P and synthetic accessibility, as well as properties that consider the 3D conformation of the ligand in the binding pocket, such as docking score and hydrogen bonding (FIGS. 9A to 9C).

FRAME excelled at producing ligands similar to the reference ligand in simple chemical features, such as formal charge, number of rings, and number of rotatable bonds (FIG. 9A). Molecules generated by FRAME had a higher median synthetic complexity score than reference ligands, which may indicate that the connections between fragments are not always indicative of known ligands (FIG. 9A). The median docking scores of the generated molecules (−8) were slightly higher than those of the reference ligands (−9), although better than those of randomly generated ligands (−6) (FIG. (A). Based on this data, the docking score is heavily influenced by key interactions such as the number of hydrogen bonds and salt-bridges.

Molecules generated using physics-based scoring functions tended to have very favorable docking scores, but were more charged and polar than reference ligands (FIG. 9A). This is unsurprising given that this property is assessed by the same scoring function used to greedily select actions, which tends to select for polar interactions. The low docking scores likely do not translate to higher binding affinity, as the excess charges and polar groups will strengthen interactions with water relative to the pocket.

### FRAME Learns Chemical Interactions

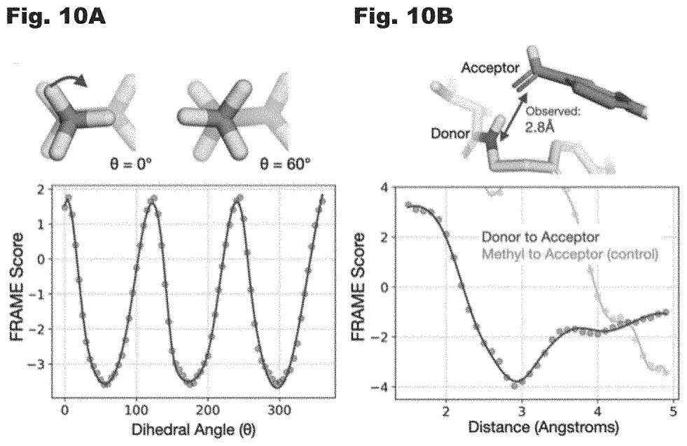

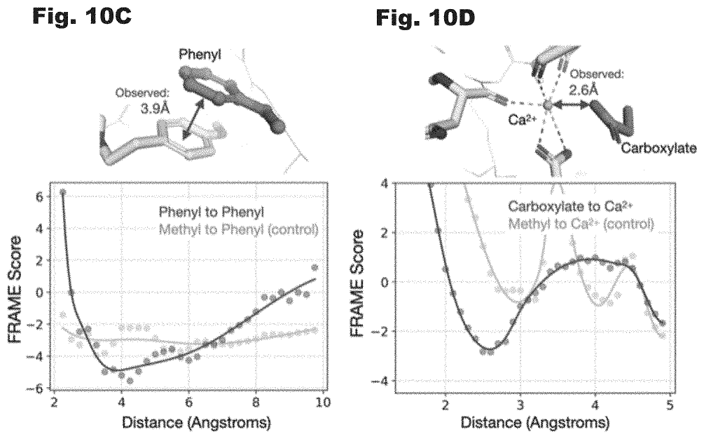

E(3) Equivariant Neural Networks are capable of learning physical principles from small sets of molecular structures. By learning these principles instead of memorizing specific atom arrangements, the models can generalize to unseen structures. The analysis of the FRAME fragment scoring function, illustrated in several examples, suggests that it can also learn such principles (FIGS. 10A to 10D).

First, it was examined whether FRAME could identify energetically favorable ligand conformations by using it to score a series of structures that varied only in the dihedral angle of a methyl group. FRAME produced a sinusoidal function that aligned with chemical intuition; the most energetically favorable conformations scored the lowest, corresponding to the staggered conformation, while the least favorable scored the highest, corresponding to the eclipsed conformation (FIG. 10A).

FRAME also recognized intermolecular interactions including hydrogen bonding and pi-pi stacking. First, the distance between a hydrogen-bond donor on the protein and acceptor atom on the fragment was varied by pulling the fragment away from the pocket and scored the resulting conformations (FIG. 10B). The minimum FRAME score corresponded to a donor-acceptor distance of 2.9 Å, precisely within the expected range of typical hydrogen bonds. The curve resembled a potential well, increasing steeply as the atoms were brought closer together and leveling off as they were pulled apart. To confirm this was specific to fragments with acceptors, the test was repeated with a non-polar methyl. This fragment had much less favorable scores and an altered minimum, showing that the model specifically recognized the interactions between the donor and acceptor atoms. The same type of experiment was performed with two aromatic rings (FIG. 10C). FRAME identified a ring centroid distance of about 4 Å as optimal, consistent with the geometry of typical pi-pi interactions in proteins. Additionally, FRAME recognizes metal-ligand interactions despite their rarity in the dataset. For example, the optimal distance between a calcium ion and carboxylate group as suggested by FRAME matches the distance observed in the unseen reference structure (FIG. 10D). These examples demonstrate the ability of the model to generalize and learn fundamental physical principles from small sets of molecular structures.

### Robustness to Input Perturbations

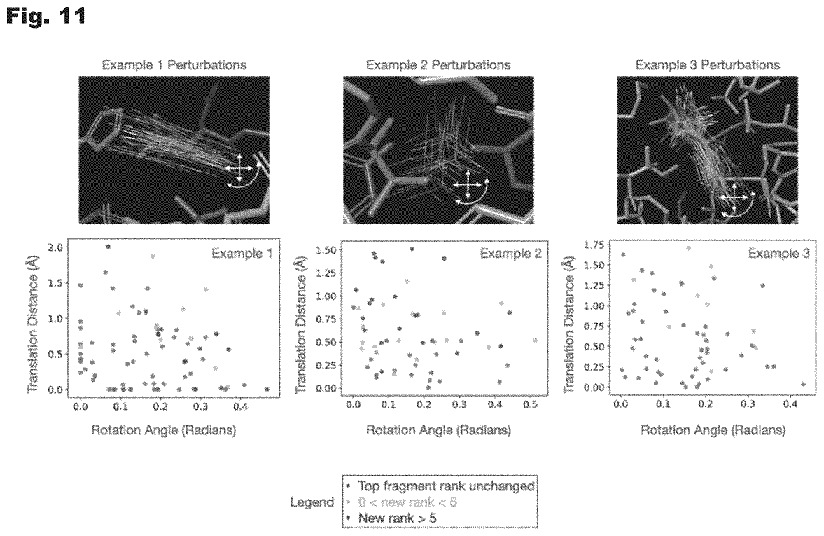

The robustness of FRAME to perturbations of the input fragment was also explored. FRAME takes as input a seed fragment placed in the protein pocket; this fragment is treated as rigid throughout the expansion process. Small random translations and rotations of the input fragment was performed on several examples to observe the effect on scoring and fragment selection (FIG. 11). Results showed that the fragment scoring model was generally robust to perturbations with translation distances less than 0.5 Å and rotations less than 10 degrees. It was found that larger perturbations could be addressed by performing a restrained force-field minimization of the seed and attached candidate fragments prior to model scoring. This suggests that the connected fragments of a ligand tend to occupy low energy minima that are recoverable if the perturbation is smaller.

### Dataset Preparation

Protein-ligand complexes were collected from PDBbind v2019. Structures were prepared using the Schrodinger suite (v2019-2); missing sidechains were added, bond orders were determined, hydrogens were added, far waters were deleted, and protonation state was determined using Epik at pH 7.0+/−2.0. Energy minimization was performed with non-hydrogen atoms constrained to an RMSD of less than 0.3 Å from the initial structure.

Ligands were then filtered to enrich drug-like small molecules. The following criteria were used as filters: molecular weight greater than 150, number of amides/molecular weight greater than 0.0057, number of rotatable bonds less than 25. Using substructure matching with rdkit, sugars, nucleotides, lipids, and duplicates were removed.

### Model Architecture

The architecture has two main components: (1) embedding unit and (2) aggregator unit.

The embedding unit consists of two layers of sequential application of self-interaction, point-convolution, self-interaction, nonlinearity, and point normalization. For the E(3) equivariant layers of the embedding unit, the maximum filter rotation order was restricted to I=2. At each point convolution, it updates the features associated to a given point p based on the features of 50 closest neighboring points in the Euclidian 3D space, weighted by their distances to p. These weighted distances were expressed in terms of Gaussian radial basis function (RBF) kernel, as a trainable network of two dense layers with hidden layer of size 12. The number of basis and maximum radius of the Gaussian RBF kernel determines the spatial resolution of the kernel, which we chose to be 12 and 12.0 Å respectively.

Starting from one hot encoding of the basic element type and ligand/fragment flags as feature channels at input, the first layer of the embedding unit mixes those features and outputs 24 feature channels per rotation order (I=0, I=1, and I=2). The second layer of the embedding unit further mixes those features to output 12 feature channels per rotation order. The 0-th rotation order outputs of this layer are then averaged across all points to be passed as input to the aggregator unit.

The learned embedding vectors of the final layer of the embedding unit act as input to the aggregator unit. The aggregator unit consists of 2 fully connected (FC) layers (followed by ELU activation function except for the final FC layer) with hidden dimension of 256. The aggregation process differs depending on the type of actions being evaluated. To score a candidate fragment at a given location, the average of the learned feature vectors was taken over all points to give one mean feature vector, which is then passed to the aggregator unit. The output of the aggregator unit is a scalar value in this case.

### Model Training

For both types of models, the training was formulated as a binary classification task with binary cross entropy loss. To address issues with imbalanced datasets, as there were considerably more negative than positive samples, the less frequent class was randomly oversampled respectively during training.

The models were trained with the Adam optimizer in Pytorch with learning rate of 0.01 and batch size of 8 for 30 epochs and monitor the loss on the validation set at every epoch. The weights of the best-performing network are then used to evaluate the predictions on the validation set. The models were trained on 1 NVIDIA Titan X GPU for 30 minutes—30 hours depending on the task.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A computational method of generating a three-dimensional ligand compound structure, comprising:
(a) selecting a target macromolecule structure and a core ligand compound structure, wherein the core ligand compound is putatively capable of associating with the target macromolecule via a chemical interaction;
(b) selecting an attachment location on the core ligand structure for addition of an atomic structure;
(c) selecting a particular atomic structure to be added at the selected attachment location; and
(d) generating a three-dimensional ligand compound structure that is the core ligand compound structure with the selected atomic structure added at the selected attachment location.

2. The method of claim 1, wherein the selecting of the attachment location is performed utilizing a computational model capable of interpreting three-dimensional data, wherein the model is trained by utilizing known ligand-macromolecule structures that are deconstructed by removing atomic structures.

3. The method of claim 2, wherein the computational model is a neural network that is equivariant to rotation and translation.

4. The method of claim 2, wherein the attachment location is at a position of a hydrogen atom.

5. The method of claim 2, wherein the attachment location is at a position of a double bond or triple bond.

6. The method of claim 2, wherein the attachment location is at a position of an atom capable of gaining a charge.

7. The method of claim 2, wherein the training is performed with supervision and binary labels are computed for each attachment location.

8. The method of claim 7, wherein the binary label computed is whether a hydrogen would be or would not be replaced by the atomic structure in the generated three-dimensional ligand compound structure.

9. The method of claim 1, wherein the selecting of the particular atomic structure is performed utilizing a computational model capable of interpreting three-dimensional data, wherein the model is trained by utilizing known ligand-macromolecule structures that are deconstructed by removing atomic structures.

10. The method of claim 9, wherein the computational model is a neural network that is equivariant to rotation and translation.

11. The method of claim 10, wherein the particular atomic structure that has been selected to be added is an atom selected from: C, O, N, P, S, H, F, Cl, or Br.

12. The method of claim 10, wherein the particular atomic structure that has been selected to be added is a small molecular structure selected from: an alkene, an alkyne, a carboxyl group, an amino groups, or a ring structure.

13. The method of claim 12, wherein the small molecular structure has between 1 and 30 atoms.

14. The method of claim 10, wherein the training is performed with supervision and labels can be computed for an additive state in which one label represents the correct additive state and a plurality of labels represents decoy states.

15. The method of claim 1, wherein the core ligand compound structure is a known ligand that has one or more atomic structures removed.

16. The method of claim 1, wherein the core ligand compound structure is a computationally generated structure that is putatively expected to associate with target macromolecule structure.

17. The method of claim 1 further comprising iteratively repeating step (b), step (c) and step (d), resulting in an addition of another selected atomic structure to the ligand structure at another selected location at each iteration, wherein step (b), step (c) and step (d) are iteratively repeated until a final three-dimensional ligand structure is yielded.

18. The method of claim 17, wherein step (b), step (c) and step (d) are iteratively repeated until the generated three-dimensional ligand compound structure reaches a particular atomic weight.

19. The method of claim 17, wherein step (b), step (c) and step (d) are iteratively repeated until a selected number of iterations are performed.

20. The method of claim 17, wherein step (b), step (c) and step (d) are iteratively repeated until the generated three-dimensional ligand compound structure achieves a particular binding affinity for the target macromolecule structure.

21. The method of claim 17, wherein step (b), step (c) and step (d) are iteratively repeated until a computational model predicts no further attachment points on the ligand structure, wherein the computational model is capable of interpreting 3D space of atomic structures and interactions between ligands and their associated macromolecule.

22. The method of claim 17 further comprising chemically synthesizing the final three-dimensional ligand structure to yield a chemically synthesized ligand.

23. The method of claim 22, wherein the chemically synthesized ligand is utilized in a medicinal formula for treatment of a medical disorder or disease; wherein the chemically synthesized ligand is utilized as modulator in biochemical experimentation; or wherein the chemically synthesized ligand is utilized in an agricultural product.
